(19) United States
(12) Patent Application Publication
(10) Pub. No.: US 2021/0244649 A1
(43) Pub. Date: Aug. 12, 2021

(54) COMPOSITION COMPRISING A SILICONE/POLYUREA OR SILICONE/POLYURETHANE COPOLYMER OR SILICONE/POLYUREA/POLYURETHANE COPOLYMER, A SILICONE RESIN, A VOLATILE ALKANE AND A C2-C8 MONOALCOHOL

(71) Applicant: L'OREAL, Paris (FR)

(72) Inventor: Guillaume KERGOSIEN, Chevilly Larue (FR)

(73) Assignee: L'OREAL, Paris (FR)

(21) Appl. No.: 17/258,630

(22) PCT Filed: Jun. 26, 2019
(86) PCT No.: PCT/EP2019/067059

§ 371 Date: Jan. 7, 2021

(30) Foreign Application Priority Data
Jul. 12, 2018 (FR) 18 56430

(51) Int. Cl. A61K 8/898; C09D 183/04; A61K 8/31; A61K 8/892; A61K 8/40; A61K 8/34; A61K 8/29; A61K 8/19; A61Q 1/02

(52) U.S. Cl. CPC A61K 8/898; C09D 183/04; A61K 8/31; A61K 8/892; A61K 2800/31; A61K 8/34; A61K 8/29; A61K 8/19; A61Q 1/02; A61K 8/40

(57) ABSTRACT

The present invention relates to a composition, especially comprising a physiologically acceptable medium, especially for coating keratin materials, more particularly for making up and/or caring for keratin materials such as the skin, and containing at least one silicone/polyurea copolymer and/or a silicone/polyurethane copolymer and/or a silicone/polyurethane copolymer of following formula (I): •b) at least one volatile alkane; and •c) at least one monoalcohol comprising from 2 to 8 carbon atoms; and •d) at least one silicone resin. The invention also relates to a process for coating keratin materials, more particularly for making up and/or caring for keratin materials such as the skin, characterized in that it comprises the application to the keratin materials of a composition as defined previously. The present invention also relates to a cosmetic assembly comprising •a composition as defined previously •a device for applying said composition to the keratin materials.

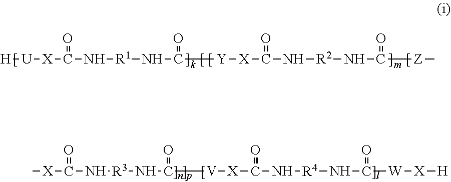

The present invention aims to propose, for the field of caring for and/or making up keratin materials, especially the skin, a novel composition comprising at least one silicone/polyurea copolymer and/or the silicone/polyurethane copolymer and/or a silicone/polyurea/polyurethane copolymer in accordance with the invention is chosen from compounds having the chemical structure of formula (I) as defined below, at least one silicone resin, at least one volatile alkane and at least one monoalcohol comprising from 2 to 8 carbon atoms.

Cosmetic makeup compositions are commonly used to give keratin materials such as the skin an esthetic color, but also to enhance the beauty of irregular skin, by making it possible to hide marks and dyschromias, and to reduce the visibility of relief imperfections such as pores and wrinkles. Many formulations have been developed to date.

For several years, consumers have been searching for compositions which provide a good wear property of the make-up effect over time in order to be able to avoid having to re-apply the composition too often. A poor wear property over time may be reflected in particular by poor wearing of the color and/or poor retention of matteness over time. This poor wear property may be characterized by a modification in the color (change or fading of the color) or the matteness following an interaction with the sebum and/or sweat secreted by the skin in the cases of foundation and pressed powder. This obliges the user to reapply the makeup very regularly, which may be time consuming.

For several years, consumers have been searching for “transfer-free” makeup compositions for keratin materials, which are compositions that have the advantage of forming a deposit that does not transfer, at least partly, onto supports with which they come into contact (glass, clothing, cigarette or fabrics).

Patent application WO 2017/216475 proposes makeup compositions comprising, in a physiologically acceptable medium, at least one silicone-polyurethane polymer such as the polymer with the INCI name: Bis-Hydroxypropyl Dimethicone/SMDI Copolymer (SILMER UR-5050® by Siltech), a silicone resin and isododecane, with a view to obtaining good makeup properties, a good wear property and good resistance to rubbing. However, some of these compositions may produce, after application, a tacky effect which is unpleasant for the consumer's comfort.

There is thus the need to find new cosmetic compositions which have a good compromise between wear property, being transfer-free and significantly reducing or even eliminating the tacky effect after application.

The Applicant has just discovered, surprisingly, that this objective could be achieved with a composition, especially a composition for coating keratin materials, more particularly for making up and/or caring for keratin materials such as the skin, and comprising, in particular in a cosmetically acceptable medium:

a) at least one silicone/polyurea copolymer and/or a silicone/polyurethane copolymer and/or a silicone/polyurea/polyurethane copolymer of formula (I) as defined below; and

b) at least one volatile alkane; and

c) at least one monoalcohol comprising from 2 to 8 carbon atoms; and

d) at least one silicone resin.

Indeed, the Applicant has observed, during its research, that the composition according to the invention results in compositions which have an excellent wear property and a good transfer-free effect while at the same time reducing or even eliminating its tacky nature.

The invention also relates to a process for coating keratin materials, more particularly for making up and/or caring for keratin materials such as the skin, characterized in that it comprises the application to the keratin materials of a composition as defined previously.

The present invention also relates to a cosmetic assembly comprising
a composition comprising, in a cosmetically acceptable medium:

a) at least one silicone/polyurea copolymer and/or a silicone/polyurethane copolymer and/or a silicone/polyurea/polyurethane copolymer of formula (I) as defined below; and

b) at least one volatile alkane; and

c) at least one monoalcohol comprising from 2 to 8 carbon atoms; and

d) at least one silicone resin
a device for applying said composition to the keratin materials.

This discovery forms the basis of the present invention.

Other characteristics, aspects and advantages of the invention will become apparent on reading the detailed description that follows.

### Definitions

In the context of the present invention, the term “keratin material” is intended to mean in particular the skin (of the body, face, around the eyes, or the eyelids).

The term “physiologically acceptable” is intended to mean compatible with the skin and/or its integuments, which has a pleasant color, odor and feel, and which does not cause any unacceptable discomfort (stinging or tautness) liable to discourage the consumer from using this composition.

Silicone/Polyurea Copolymer, Silicone/Polyurethane Copolymer, Silicone/Polyurea/Polyurethane Copolymer

The silicone/polyurea copolymer and/or the silicone/polyurethane copolymer and/or the silicone/polyurea/polyurethane copolymer in accordance with the invention is chosen from compounds having the chemical structure of following formula (I):

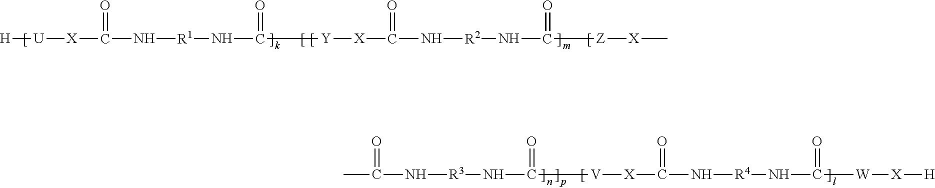

in which:

the radicals R¹, R², R³, R⁴, which are identical or different, represent a linear or branched, acyclic or cyclic, aliphatic or aromatic C₆-C₂₀ divalent hydrocarbon-based group;

k and l are numbers ranging from 0 to 100,

m is a number ranging from 0 to 100, preferably ranging from 1 to 100,

n and p are numbers ranging from 1 to 100,

X denotes an oxygen (O) or nitrogen (N) atom,

when X is an oxygen atom (O), m ranges from 1 to 100,

when X is a nitrogen atom (N), m ranges from 0 to 100,

Y denotes the following formula (II):

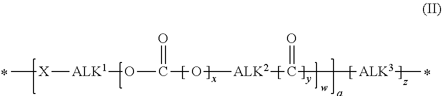

in which a is a number ranging from 1 to 100,

w is a number ranging from 0 to 100,

x, y and z, which are identical or different, are 0 or 1, and

when x=0, then w=1 and y=1 and z=1

when x=1, then y=0 and z=0

X represents an oxygen atom (0) or a nitrogen atom (N),

ALK¹, ALK², ALK³, which are identical or different, represent a linear or branched, acyclic or cyclic, aliphatic or aromatic C₁-C₂₀ divalent hydrocarbon-based group, preferably:
i) a *—(CH₂)_{c}—* group in which c is a number between 1 and 20, preferably between 1 and 6, or
ii) a *—CH(CH₃)—CH₂—* group

Z denotes the following formula (III)

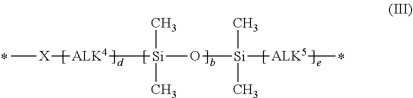

in which:

b is a number ranging from 1 to 1000,

d and e, which are identical or different, are 0 or 1, and X represents an oxygen (O) or nitrogen (N) atom,

ALK⁴, ALK⁵, which are identical or different, represent a *—(CH₂)_{c}—* group as defined previously;

U, V, W represent the formula (II) or the formula (III) as defined previously.

Among the radicals R¹, R², R³, R⁴ of aromatic type, mention may be made of the toluene, diphenylmethylene, dibenzyl or xylylene groups.

Among the radicals R¹, R², R³, R⁴ of cyclic type, mention may be made of the 4,4′-dicyclohexylmethane or isophorone groups.

Among the radicals R¹, R², R³, R⁴, mention may be made of the hexamethylene group.

The silicone/polyurea copolymer according to the invention may especially be obtained by copolymerization of a diisocyanate with an α,ω-aminopolysiloxane. Silicone/polyurea copolymers of INCI name BIS-AMINOPROPYL DIMETHICONE/IPDI COPOLYMER or of INCI name POLYUREADIMETHICONE are especially sold under the reference Belsil® UD 60, Belsil® UD 80, Wacker-Belsil® UD 140, and Belsil® UD 200 by Wacker.

The silicone/polyurethane copolymer according to the invention may especially be obtained by copolymerization of a diisocyanate with an α,ω-hydroxypolysiloxane and a diol preferably chosen from polyether diols, polyester diols and polycarbonate diols. Silicone/polyurethane copolymers are especially sold under the trade names CarboSil® TSPCU and PurSil® TSPU by DSM.

The copolymer according to the invention may also be a silicone/polyurea/polyurethane copolymer. It is especially obtained by copolymerization of a diisocyanate with an α,ω-aminopolysiloxane and an α,ω-hydroxypolysiloxane.

Use will more particularly be made of a silicone/polyurea copolymer of the copolymer of bis-aminopropyl dimethicone and of isophorone diisocyanate type with the INCI name: BIS-AMINOPROPYL DIMETHICONE/IPDI COPOLYMER BIS-AMINOPROPYL DIMETHICONE/IPDI COPOLYMER, especially sold under the trade name Belsil® UD 80 by Wacker.

The copolymer(s) is (are) preferably present in the composition in a content ranging from 1% to 20% by weight, better still from 3% to 15% by weight, preferably from 5% to 10% by weight relative to the total weight of said composition.

Volatile Alkanes

The compositions of the invention comprise at least one linear or branched volatile alkane.

The term “alkane” is intended to mean any compound comprising a linear or branched, saturated, hydrocarbon-based chain constituted exclusively of carbon atoms and hydrogen atoms.

The term “volatile alkane” that is suitable for use in the invention is intended to mean a cosmetic alkane, which is capable of evaporating on contact with the skin in less than one hour, at ambient temperature (25° C.) and atmospheric pressure (760 mmHg, i.e. 101 325 Pa), which is liquid at ambient temperature, especially having an evaporation rate ranging from 0.01 to 15 mg/cm²/minute, at ambient temperature (25° C.) and atmospheric pressure (760 mmHg).

Among the volatile alkanes in accordance with the invention, mention may be made is of:

Volatile linear alkanes;

Volatile branched alkanes;

and mixtures thereof.

a) Volatile Linear Alkanes

The volatile linear alkanes that are suitable for the invention are preferably chosen from volatile linear alkanes comprising from 7 to 14 carbon atoms.

As examples of alkanes that are suitable for the invention, mention may be made of the alkanes described in patent applications WO 2007/068371 and WO 2008/155059 from the company Cognis (mixtures of distinct alkanes differing by at least one carbon). These alkanes are obtained from fatty alcohols, which are themselves obtained from copra oil or palm oil.

As examples of linear alkanes that are suitable for the invention, mention may be made of n-heptane (C₇), n-octane (C₈), n-nonane (C₉), n-decane (C₁₀), n-undecane (C₁₁), n-dodecane (C₁₂), n-tridecane (C₁₃) and n-tetradecane (C₁₄), and mixtures thereof.

According to one preferred mode, mention may be made of mixtures of n-undecane (C₁₁) and of n-tridecane (C₁₃) obtained in Examples 1 and 2 of patent application WO 2008/155059 from the company Cognis.

Mention may also be made of n-dodecane (C₁₂) and n-tetradecane (C₁₄) sold by Sasol under the respective references PARAFOL® 12 97 and PARAFOL® 14 97, and also mixtures thereof.

b) Volatile Branched Alkanes

Among the volatile branched alkanes, mention may be made of branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, and mixtures thereof. Isododecane will preferably be used.

The volatile alkane(s) are preferably present in the composition in concentrations ranging from 35% to 95% by weight and more preferentially from 50% to 80% by weight relative to the total weight of the composition.

Silicone Resin

The composition according to the invention comprises at least one silicone resin.

More generally, the term “resin” means a compound of which the structure is three-dimensional. “Silicone resins” are also known as “siloxane resins”. Thus, for the purposes of the present invention, a polydimethylsiloxane is not a silicone resin.

The nomenclature of silicone resins (also known as siloxane resins) is known under the name “MDTQ”, the resin being described as a function of the various siloxane monomer units it comprises, each of the letters “MDTQ” characterizing a type of unit.

The letter M represents the monofunctional unit of formula R1R2R3SiO_{1/2}, the silicon atom being bonded to only one oxygen atom in the polymer comprising this unit.

The letter D means a difunctional unit R1R2SiO_{2/2} in which the silicon atom is bonded to two oxygen atoms.

The letter T represents a trifunctional unit of formula R1SiO_{3/2}.

Such resins are described, for example, in the Encyclopedia of Polymer Science and Engineering, vol. 15, John Wiley and Sons, New York, (1989), pp. 265-270, and U.S. Pat. Nos. 2,676,182, 3,627,851, 3,772,247, 5,248,739 or else U.S. Pat. Nos. 5,082,706, 5,319,040, 5,302,685 and 4,935,484.

In the units M, D and T defined previously, R, namely R1, R2 and R3, represents a hydrocarbon-based radical (especially alkyl) containing from 1 to 10 carbon atoms, a phenyl group, a phenylalkyl group or a hydroxyl group.

Finally, the letter “Q” means a tetrafunctional unit SiO_{4/2} in which the silicon atom is bonded to four oxygen atoms, which are themselves bonded to the rest of the polymer.

Various silicone resins with different properties may be obtained from these different units, the properties of these polymers varying as a function of the type of monomer (or unit), the nature and number of the radical R, the length of the polymer chain, the degree of branching and the size of the side chains.

As silicone resins that may be used in the compositions according to the invention, use may be made, for example, of silicone resins of MQ type, of T type or of MQT type.

MQ Resins:

As examples of silicone resins of MQ type, mention may be made of the alkyl siloxysilicates of formula [(R1)₃SiO_{1/2}]ₓ(SiO)_{y} (MQ units) in which x and y are integers ranging from 50 to 80, and such that the group R1 represents a radical as defined previously, and is preferably an alkyl group containing from 1 to 8 carbon atoms or a hydroxyl group, preferably a methyl group.

As examples of solid silicone resins of MQ type of trimethyl siloxysilicate type, mention may be made of those sold under the reference SR1000®, E 1 170-002® or SS 4230® by the company General Electric, under the reference TMS 803®, WACKER 803® and 804® by the company Wacker, under the name “KF-7312J®” by the company Shin-Etsu, “DC 749®”, “DC 593®” by the company Dow Corning.

As silicone resins comprising MQ siloxysilicate units, mention may also be made of phenylalkylsiloxysilicate resins, such as phenylpropyldimethylsiloxysilicate (Silshine 151® sold by the company General Electric). The preparation of such resins is described especially in patent U.S. Pat. No. 5,817,302.

T Resins:

Examples of silicone resins of type T that may be mentioned include the polysilsesquioxanes of formula (RSiO_{3/2})ₓ (T units) in which x is greater than 100 and such that the group R is an alkyl group containing from 1 to 10 carbon atoms, said polysilsesquioxanes also possibly comprising Si—OH end groups.

Polymethylsilsesquioxane resins that may preferably be used are those in which R represents a methyl group, for instance those sold:
by the company Wacker under the reference Resin MK®, such as Belsil PMS MK®: polymer comprising CH₃SiO_{3/2} repeating units (T units), which may also comprise up to 1% by weight of (CH₃)₂SiO_{2/2} units (D units) and having an average molecular weight of about 10 000 g/mol, or
by the company Shin-Etsu under the references KR-220L®, which are composed of T units of formula CH₃SiO_{3/2} and contain Si—OH (silanol) end groups, under the reference KR-242A®, which comprise 98% of T units and 2% of dimethyl D units and contain Si—OH end groups, or else under the reference KR-251®, comprising 88% of T units and 12% of dimethyl D units and contain Si—OH end groups.

MQT Resins:

Resins comprising MQT units that are especially known are those mentioned in U.S. Pat. No. 5,110,890.

A preferred form of resins of MQT type are MQT-propyl (also known as MQTpr) resins. Such resins that may be used in the compositions according to the invention are especially the resins described and prepared in patent application WO 2005/075 542, the content of which is incorporated herein by reference.

The MQ-T-propyl resin preferably comprises the following units:
(i) (R1₃SiO_{1/2})ₐ
(ii) (R2₂SiO_{2/2})_{b}
(iii) (R3SiO_{3/2})_{c} and
(iv) (SiO_{4/2})_{d}

With R1, R2 and R3 independently representing a hydrocarbon-based radical (especially alkyl) containing from 1 to 10 carbon atoms, a phenyl group, a phenylalkyl group or a hydroxyl group and preferably an alkyl radical containing from 1 to 8 carbon atoms or a phenyl group,

a, b, c and d being mole fractions,

a being between 0.05 and 0.5,

b being between 0 and 0.3,

c being greater than zero,

d being between 0.05 and 0.6,

a+b+c+d=1,

on condition that more than 40 mol % of the groups R3 of the siloxane resin are propyl groups.

Preferably, the siloxane resin comprises the units:
(i) (R1₃SiO_{1/2})ₐ
(iii) (R3SiO_{3/2})_{c} and
(iv) (SiO_{4/2})_{d}

With R1 and R3 independently representing an alkyl group containing from 1 to 8 carbon atoms, R1 preferably being a methyl group and R3 preferably being a propyl group,

a being between 0.05 and 0.5 and preferably between 0.15 and 0.4,

c being greater than zero, preferably between 0.15 and 0.4,

d being between 0.05 and 0.6, preferably between 0.2 and 0.6 or alternatively between 0.2 and 0.55,

a+b+c+d=1, and a, b, c and d being mole fractions, on condition that more than 40 mol % of the groups R3 of the siloxane resin are propyl groups.

The siloxane resins that may be used according to the invention may be obtained via a process comprising the reaction of:

A) an MQ resin comprising at least 80 mol % of units (R1₃SiO_{1/2})ₐ and (SiO_{4/2})_{d},

R1 representing an alkyl group containing from 1 to 8 carbon atoms, an aryl group, a carbinol group or an amino group,

a and d being greater than zero,

the ratio a/d being between 0.5 and 1.5; and

B) a T-propyl resin comprising at least 80 mol % of units (R3SiO_{3/2})_{c},

R3 representing an alkyl group containing from 1 to 8 carbon atoms, an aryl group, a carbinol group or an amino group,

c being greater than zero,

on condition that at least 40 mol % of the groups R3 are propyl groups,

in which the weight ratio A/B is between 95/5 and 15/85 and preferably the weight ratio NB is 30/70.

Advantageously, the weight ratio NB is between 95/5 and 15/85. Preferably, the ratio NB is less than or equal to 70/30. These preferred ratios have proven to afford comfortable deposits.

Preferably, the composition according to the invention comprises, as silicone resin, at least one MQ resin as described previously, and more particularly of trimethylsiloxysilicate type.

According to a particular embodiment of the invention, the silicone resin is present in the composition in a resin solids content ranging from 2% to 20% by weight relative to the total weight of the composition, preferably ranging from 3% to 15% by weight relative to the weight of the composition.

Monoalcohol

The compositions of the invention comprise at least one monoalcohol comprising from 2 to 8 carbon atoms, especially from 2 to 6 carbon atoms and in particular from 2 to 4 carbon atoms.

The compositions of the invention may comprise one or more monoalcohol(s).

This monoalcohol can be represented, for example, by the formula RaOH, in which Ra represents a linear or branched alkyl group comprising from 2 to 8 carbon atoms.

As monoalcohol, mention may be made of ethanol, isopropanol, propanol or butanol, and mixtures thereof.

According to one embodiment, the compositions of the invention comprise isopropanol.

The amount of monoalcohol(s) preferentially ranges from 2% to 20% by weight and more preferentially from 3% to 15% by weight relative to the total weight of said composition.

Pigments

According to a particular embodiment of the invention, the composition according to the invention also comprises at least one pigment.

The term “pigments” means white or colored, mineral or organic particles, which are insoluble in an aqueous medium, and which are intended to color and/or opacify the resulting composition and/or deposit. These pigments may be white or colored, and mineral and/or organic.

Preferably, the composition comprises at least 5% by weight of pigments, more preferentially from 5% to 40% by weight of pigments, in particular from 10% to 30% by weight and preferably from 10% to 20% by weight of pigments, relative to the total weight of said composition.

According to a particular embodiment, the pigments used according to the invention are chosen from mineral pigments.

The term “mineral pigment” refers to any pigment that satisfies the definition in Ullmann's encyclopedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminum powder or copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The size of the pigment that is useful in the context of the present invention is generally greater than 100 nm and may range up to 10 μm, preferably from 200 nm to 5 μm and more preferentially from 300 nm to 1 μm.

According to a particular form of the invention, the pigments have a size characterized by a D[50] greater than 100 nm and possibly ranging up to 10 μm, preferably from 200 nm to 5 μm and more preferentially from 300 nm to 1 μm.

The sizes are measured by static light scattering using a commercial MasterSizer 3000 particle size analyzer from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicron to multimicron; it allows an “effective” particle diameter to be determined. This theory is especially described in the publication by Van de Hulst, H. C., Light Scattering by Small Particles, Chapters 9 and 10, Wiley, New York, 1957.

D[50] represents the maximum size that 50% by volume of the particles have.

In the context of the present invention, the mineral pigments are more particularly chosen from iron oxides and/or titanium dioxides, and more preferentially iron oxide and/or titanium dioxide pigments coated with at least one lipophilic or hydrophobic compound.

Examples that may be mentioned more particularly include titanium dioxides and iron oxide coated with aluminum stearoyl glutamate, sold, for example, under the reference NAI® by the company Miyoshi Kasei.

As mineral pigments that may be used in the invention, mention may also be made of nacres.

The term “nacres” should be understood as meaning colored particles of any form, which may or may not be iridescent, especially produced by certain molluscs in their shell, or alternatively synthesized, and which have a color effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic colorants.

Examples of nacres that may also be mentioned include natural mica coated with titanium oxide, with iron oxide, with natural pigment or with bismuth oxychloride. Among the nacres available on the market, mention may be made of the nacres Timica®, Flamenco® and Duochrome® (based on mica) sold by the company Engelhard, the Timiron® nacres sold by the company Merck, the Prestige® mica-based nacres sold by the company Eckart, and the Sunshine® synthetic mica-based nacres sold by the company Sun Chemical.

The nacres may more particularly have a yellow, pink, red, bronze, orange, brown, gold and/or coppery color or tint.

As illustrations of nacres that may be used in the context of the present invention, mention may be made especially of the gold-colored nacres sold especially by the company Engelhard under the name Brilliant gold 212G (Timica®), Gold 222C (Cloisonne®), Sparkle gold (Timica®), Gold 4504 (Chromalite) and Monarch gold 233X® (Cloisonne); the bronze nacres sold especially by the company Merck under the name Bronze Fine® (17384) (Colorona®) and Bronze (17353) (Colorona®) and by the company Engelhard under the name Super bronze (Cloisonne®); the orange nacres sold especially by the company Engelhard under the name Orange 363C (Cloisonne®) and Orange MCR 101 (Cosmica®) and by the company Merck under the name Passion orange (Colorona) and Matte orange (17449) (Microna); the brown nacres sold especially by the company Engelhard under the name Nu-antique copper 340XB (Cloisonne®) and Brown CL4509 (Chromalite®); the nacres with a copper tint sold especially by the company Engelhard under the name Copper 340A (Timica®); the nacres with a red tint sold especially by the company Merck under the name Sienna fine (17386) (Colorona®); the nacres with a yellow tint sold especially by the company Engelhard under the name Yellow (4502) (Chromalite®); the red nacres with a gold tint sold especially by the company Engelhard under the name Sunstone G012 (Gemtone®); the pink nacres sold especially by the company Engelhard under the name Tan opale G005 (Gemtone®); the black nacres with a gold tint sold especially by the company Engelhard under the name Nu antique bronze 240 AB (Timica®), the blue nacres sold especially by the company Merck under the name Matte blue (17433) (Microna), the white nacres with a silvery tint sold especially by the company Merck under the name Xirona Silver®, and the golden-green pink-orange nacres sold especially by the company Merck under the name Indian summer (Xirona®), and mixtures thereof.

Among the pigments that may be used according to the invention, mention may also be made of those having an optical effect different from a simple conventional coloring effect, i.e. a unified and stabilized effect such as produced by conventional colorants, for instance monochromatic pigments. For the purposes of the invention, the term “stabilized” means lacking the effect of variability of the color with the angle of observation or in response to a temperature change.

For example, this material may be chosen from particles with a metallic tint, goniochromatic coloring agents, diffractive pigments, thermochromic agents, optical brighteners, and also fibers, in particular interference fibers. Needless to say, these various materials may be combined in order simultaneously to afford two effects, or even a novel effect in accordance with the invention.

The particles with a metallic tint that may be used in the invention are in particular chosen from:
particles of at least one metal and/or of at least one metal derivative,
particles comprising a monomaterial or multimaterial organic or mineral substrate, at least partially coated with at least one layer with a metallic tint comprising at least one metal and/or at least one metal derivative, and mixtures of said particles.

Among the metals that may be present in said particles, mention may for example be made of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo, Cr and mixtures or alloys thereof (for example, bronzes and brasses) are preferred metals.

The term “metal derivatives” denotes compounds derived from metals, especially oxides, fluorides, chlorides and sulfides.

Illustrations of these particles that may be mentioned include aluminum particles, such as those sold under the names Starbrite 1200 EAC® by the company Silberline and Metalure® by the company Eckart.

Mention may also be made of metal powders of copper or of alloy mixtures such as the references 2844® sold by the company Radium Bronze, metallic pigments, for instance aluminum or bronze, such as those sold under the names Rotosafe 700® from the company Eckart, silica-coated aluminum particles sold under the name Visionaire Bright Silver® from the company Eckart, and metal alloy particles, for instance the silica-coated bronze (alloy of copper and zinc) powders sold under the name Visionaire Bright Natural Gold from the company Eckart.

They may also be particles comprising a glass substrate, such as those sold by the company Nippon Sheet Glass under the name Microglass Metashine®.

The goniochromatic coloring agent may be chosen, for example, from multilayer interference structures and liquid-crystal coloring agents.

Examples of symmetrical multilayer interference structures that may be used in the compositions prepared in accordance with the invention are, for example, the following structures: Al/SiO₂/Al/SiO₂/Al, pigments having this structure being sold by the company DuPont de Nemours; Cr/MgF₂/Al/MgF₂/Cr, pigments having this structure being sold under the name Chromaflair® by the company Flex; MOS₂/SiO₂/Al/SiO₂/MOS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of the Infinite Colors® pigments from the company Shiseido. Depending on the thickness and the nature of the various layers, different effects are obtained. Thus, with the Fe₂O₃/SiO₂/Al/SiO₂/Fe₂O₃ structure, the color changes from greenish gold to reddish grey for SiO₂ layers of 320 to 350 nm; from red to gold for SiO₂ layers of 380 to 400 nm; from violet to green for SiO₂ layers of 410 to 420 nm; from copper to red for SiO₂ layers of 430 to 440 nm.

As examples of pigments with a polymeric multilayer structure, mention may be made of those sold by the company 3M under the name Color Glitter®.

Examples of liquid-crystal goniochromatic particles that may be used include those sold by the company Chenix® and also the product sold under the name Helicone® HC by the company Wacker.

Hydrophobic Coated Pigments

Preferably, the compositions according to the invention comprise at least one pigment coated with at least one lipophilic or hydrophobic compound and especially as detailed below.

This type of pigment is particularly advantageous in so far as it may be considered in a large amount together with a large amount of water. What is more, in so far as they are treated with a hydrophobic compound, they show a predominant affinity for the oily gelled phase, which can then convey them.

Needless to say, the compositions according to the invention may in parallel contain uncoated pigments.

The coating may also comprise at least one additional non-lipophilic compound. For the purposes of the invention, the “coating” of a pigment according to the invention generally denotes the total or partial surface treatment of the pigment with a surface agent absorbed, adsorbed or grafted onto said pigment.

The surface-treated pigments may be prepared according to surface treatment techniques of chemical, electronic, mechanochemical or mechanical nature that are well known to those skilled in the art. Commercial products may also be used.

The surface agent may be absorbed, adsorbed or grafted onto the pigments by evaporation of solvent, chemical reaction and creation of a covalent bond.

According to one variant, the surface treatment is constituted of a coating of the pigments.

The coating may represent from 0.1% to 20% by weight and in particular from 0.5% to 5% by weight of the total weight of the coated pigment.

The coating may be realized, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be realized, for example, by chemical reaction of a surface agent with the surface of the solid pigment particles and creation of a covalent bond between the surface agent and the particles. This method is especially described in the U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited at the surface of the pigments.

Lipophilic or Hydrophobic Treatment Agent

When the pigment comprises a lipophilic or hydrophobic coating, it is preferably present in the fatty phase of the composition according to the invention.

According to a particular embodiment of the invention, the pigments may be coated according to the invention with at least one compound chosen from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

Silicone Surface Agent

According to a particular embodiment, the pigments may be totally or partially surface-treated with a compound of silicone nature. The silicone surface agents may be chosen from organopolysiloxanes, silane derivatives, silicone-acrylate copolymers, silicone resins, and mixtures thereof.

The term “organopolysiloxane compound” is intended to mean a compound having a structure comprising an alternance of silicone atoms and oxygen atoms and comprising organic radicals linked to the silicon atoms.

i) Non-Elastomeric Organopolysiloxane

Non-elastomeric organopolysiloxanes that may especially be mentioned include polydimethylsiloxanes, polymethylhydrogenosiloxanes and polyalkoxydimethylsiloxanes.

The alkoxy group may be represented by the radical R—O— such that R represents methyl, ethyl, propyl, butyl or octyl, 2-phenylethyl, 2-phenylpropyl or 3,3,3-trifluoropropyl radicals, aryl radicals such as phenyl, tolyl or xylyl, or substituted aryl radicals such as phenylethyl.

One method for surface-treating pigments with a polymethylhydrogenosiloxane consists in dispersing the pigments in an organic solvent and then in adding the silicone compound. On heating the mixture, covalent bonds are created between the silicone compound and the surface of the pigment.

According to a preferred embodiment, the silicone surface agent may be a non-elastomeric organopolysiloxane, especially chosen from polydimethylsiloxanes.

ii) Alkylsilanes and Alkoxysilanes

Silanes bearing alkoxy functionality are especially described by Witucki in “A silane primer, Chemistry and applications of alkoxy silanes, Journal of Coatings Technology, 65, 822, pages 57-60, 1993”.

Alkoxysilanes such as the alkyltriethoxysilanes and the alkyltrimethoxysilanes sold under the references Silquest A-137® (OSI Specialities) and Prosil 9202® (PCR) may be used for coating the pigments.

The use of alkylpolysiloxanes bearing a reactive end group such as alkoxy, hydroxyl, halogen, amino or imino is described in application JP H07-196946. They are also suitable for treating the pigments.

iii) Silicone-Acrylate Polymers

Grafted silicone-acrylic polymers having a silicone backbone as described in patents U.S. Pat. Nos. 5,725,882, 5,209,924, 4,972,037, 4,981,903, 4,981,902 and 5,468,477 and in patents U.S. Pat. No. 5,219,560 and EP 0 388 582 may be used.

Other silicone-acrylate polymers may be silicone polymers comprising in their structure the unit of formula (I) below:

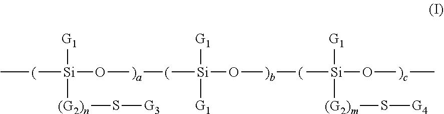

in which the radicals G₁, which may be identical or different, represent hydrogen or a C₁-C₁₀ alkyl radical or alternatively a phenyl radical; the radicals G₂, which may be identical or different, represent a C₁-C₁₀ alkylene group; G₃ represents a polymeric residue resulting from the (homo)polymerization of at least one ethylenically unsaturated anionic monomer; G₄ represents a polymeric residue resulting from the (homo)polymerization of at least one ethylenically unsaturated hydrophobic monomer; m and n are equal to 0 or 1; a is an integer ranging from 0 to 50; b is an integer that may be between 10 and 350, c is an integer ranging from 0 to 50; with the proviso that one of the parameters a and c is other than 0.

Preferably, the unit of formula (I) above has at least one, and even more preferentially all, of the following characteristics:
the radicals G1 denote an alkyl radical, preferably a methyl radical;
n is non-zero, and the radicals G2 represent a divalent C1-C3 radical, preferably a propylene radical;
G3 represents a polymeric radical resulting from the (homo)polymerization of at least one monomer of the ethylenically unsaturated carboxylic acid type, preferably acrylic acid and/or methacrylic acid;
G4 represents a polymeric radical resulting from the (homo)polymerization of at least one monomer of the (C1-C10)alkyl (meth)acrylate type, preferably of the isobutyl or methyl (meth)acrylate type.

Examples of silicone polymers corresponding to formula (I) are especially polydimethylsiloxanes (PDMS) onto which are grafted, via a connecting chain unit of thiopropylene type, mixed polymer units of the poly(meth)acrylic acid type and of the polymethyl (meth)acrylate type.

Other examples of silicone polymers corresponding to formula (I) are especially polydimethylsiloxanes (PDMS) onto which are grafted, via a connecting chain unit of thiopropylene type, polymer units of the polyisobutyl (meth)acrylate type.

iv) Silicone Resins

The silicone surface agent may be chosen from the silicone resins as defined previously.

Fluoro Surface Agent

The pigments may be totally or partially surface-treated with a compound of fluoro nature.

The fluoro surface agents may be chosen from perfluoroalkyl phosphates, perfluoropolyethers, polytetrafluoropolyethylenes (PTFE), perfluoroalkanes, perfluoroalkyl silazanes, polyhexafluoropropylene oxides, and polyorganosiloxanes comprising perfluoroalkyl perfluoropolyether groups.

The term “perfluoroalkyl radical” is intended to mean an alkyl radical in which all the hydrogen atoms have been replaced with fluorine atoms.

Perfluoropolyethers are especially described in patent application EP 0 486 135, and sold under the trade name Fomblin® by the company Montefluos.

Perfluoroalkyl phosphates are in particular described in application JP H05-86984. The perfluoroalkyl diethanolamine phosphates sold by Asahi Glass under the reference AsahiGuard AG530® may be used.

Among the linear perfluoroalkanes that may be mentioned are perfluorocycloalkanes, perfluoro(alkylcycloalkanes), perfluoropolycycloalkanes, aromatic perfluoro hydrocarbons (perfluoroarenes) and hydrocarbon-based perfluoro organic compounds comprising at least one heteroatom.

Among the perfluoroalkanes, mention may be made of the linear alkane series such as perfluorooctane, perfluorononane or perfluorodecane.

Among the perfluorocycloalkanes and perfluoro(alkylcycloalkanes), mention may be made of perfluorodecalin sold under the name Flutec PP5 GMP® by the company Rhodia, perfluoro(methyldecalin) and perfluoro(C3-C5 alkylcyclohexanes) such as perfluoro(butylcyclohexane).

Among the perfluoropolycycloalkanes, mention may be made of bicyclo[3.3.1]nonane derivatives such as perfluorotrimethylbicyclo[3.3.1]nonane, adamantane derivatives such as perfluorodimethyladamantane, and hydrogenated perfluorophenanthrene derivatives such as tetracosafluorotetradecahydrophenanthrene.

Among the perfluoroarenes, mention may be made of perfluoronaphthalene derivatives, for instance perfluoronaphthalene and perfluoromethyl-1-naphthalene.

As examples of commercial references of pigments treated with a fluoro compound, mention may be made of:
yellow iron oxide/perfluoroalkyl phosphate sold under the reference PF 5 Yellow 601® by the company Daito Kasei;
red iron oxide/perfluoroalkyl phosphate sold under the reference PF 5 Red R 516L® by the company Daito Kasei;
black iron oxide/perfluoroalkyl phosphate sold under the reference PF 5 Black BL100® by the company Daito Kasei;
titanium dioxide/perfluoroalkyl phosphate sold under the reference PF 5 TiO2 CR 50® by the company Daito Kasei;
yellow iron oxide/perfluoropolymethyl isopropyl ether sold under the reference Iron oxide yellow BF-25-3® by the company Toshiki;
DC Red 7/perfluoropolymethyl isopropyl ether sold under the reference D&C Red 7 FHC® by the company Cardre Inc.; and
DC Red 6/PTFE® sold under the reference T 9506® by the company Warner-Jenkinson.

Fluorosilicone Surface Agent

The pigments may be totally or partially surface-treated with a compound of fluorosilicone nature.

The fluorosilicone compound may be chosen from perfluoroalkyl dimethicones, perfluoroalkyl silanes and perfluoroalkyl trialkoxysilanes.

Perfluoroalkyl silanes that may be mentioned include the products LP-IT® and LP-4T® sold by Shin-Etsu Silicone.

The perfluoroalkyl dimethicones may be represented by the following formula:

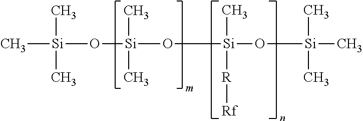

in which:
R represents a linear or branched divalent alkyl group containing from 1 to 6 carbon atoms, preferably a divalent methyl, ethyl, propyl or butyl group;
Rf represents a perfluoroalkyl radical containing 1 to 9 carbon atoms and preferably 1 to 4 carbon atoms;
m is chosen between 0 and 150 and preferably between 20 and 100; and
n is chosen between 1 and 300 and preferably between 1 and 100.

As examples of commercial references of pigments treated with a fluorosilicone compound, mention may be made of titanium dioxide/fluorosilicone sold under the reference Fluorosil Titanium dioxide 100TA® by the company Advanced Dermaceuticals International Inc.

Other Lipophilic Surface Agents

The hydrophobic treating agent may also be chosen from:

i) metal soaps such as aluminum dimyristate and the aluminum salt of hydrogenated tallow glutamate.

Metal soaps that may especially be mentioned include metal soaps of fatty acids containing from 12 to 22 carbon atoms and in particular those containing from 12 to 18 carbon atoms.

The metal of the metal soap may especially be zinc or magnesium.

Metal soaps that may be used include zinc laurate, magnesium stearate, magnesium myristate and zinc stearate, and mixtures thereof;

ii) fatty acids such as lauric acid, myristic acid, stearic acid and palmitic acid;

iii)N-acylamino acids or salts thereof, which may comprise an acyl group containing from 8 to 22 carbon atoms, for instance a 2-ethylhexanoyl, caproyl, lauroyl, myristoyl, palmitoyl, stearoyl or cocoyl group;

The amino acid may be, for example, lysine, glutamic acid or alanine.

The salts of these compounds may be the aluminum, magnesium, calcium, zirconium, zinc, sodium or potassium salts.

Thus, according to a particularly preferred embodiment, an N-acylamino acid derivative may in particular be a glutamic acid derivative and/or a salt thereof, and more particularly a stearoyl glutamate, for instance aluminum stearoyl glutamate.

iv) lecithin and derivatives thereof;

v) isopropyl triisostearyl titanate.

As examples of isopropyl titanium triisostearate (ITT)-treated pigments, mention may be made of those sold under the commercial references BWBO-I2 (Iron oxide CI77499 and isopropyl titanium triisostearate), BWYO-I2 (Iron oxide CI77492 and isopropyl titanium triisostearate) and BWRO-I2 (Iron oxide CI77491 and isopropyl titanium triisostearate) by the company Kobo;

vi) isostearyl sebacate;

vii) natural plant or animal waxes or polar synthetic waxes;

viii) fatty esters, in particular jojoba esters;

ix) phospholipids; and

x) mixtures thereof.

The waxes mentioned in the compounds mentioned previously may be those generally used in cosmetics, as defined hereinbelow.

They may especially be hydrocarbon-based, silicone and/or fluoro waxes, optionally comprising ester or hydroxyl functions. They may also be of natural or synthetic origin

The term “polar wax” is intended to mean a wax containing chemical compounds comprising at least one polar group. Polar groups are well known to those skilled in the art; they may be, for example, alcohol, ester or carboxylic acid groups.

Polyethylene waxes, paraffin waxes, microcrystalline waxes, ozokerite and Fischer-Tropsch waxes are not included among polar waxes.

In particular, the polar waxes have a mean Hansen solubility parameter δa at 25° C. such that δa>0 (J/cm³)^{1/2} and better still δa>1 (J/cm³)^{1/2}:

δₐ=\sqrt{δₚ²+δₕ²}

in which δp and δh are, respectively, the polar contributions and contributions of interaction types specific to the Hansen solubility parameters.

The definition of solvents in the three-dimensional solubility space according to Hansen is described in the article by C. M. Hansen: “The three-dimensional solubility parameters”, J. Paint Technol. 39, 105 (1967):
δh characterizes the specific interaction forces (such as hydrogen bonding, acid/base, donor/acceptor, etc.);
δp by characterizes the Debye interaction forces between permanent dipoles and also the Keesom interaction forces between induced dipoles and permanent dipoles.

The parameters δp and δh are expressed in (J/cm³)^{1/2}.

A polar wax is especially formed from molecules comprising, besides carbon and hydrogen atoms in their chemical structure, heteroatoms (such as O, N and P).

Non-limiting illustrations of these polar waxes that may especially be mentioned include natural polar waxes, such as beeswax, lanolin wax, orange wax, lemon wax and Chinese insect waxes, rice bran wax, carnauba wax, candelilla wax, ouricury wax, cork fiber wax, sugarcane wax, Japan wax, sumac wax and montan wax.

According to a particular embodiment, the pigments may be coated with at least one compound chosen from silicone surface agents; fluoro surface agents; N-acylamino acids or salts thereof; isopropyl triisostearyl titanate; natural plant or animal waxes; fatty esters; and mixtures thereof.

According to a particularly preferred embodiment, the pigments may be coated with an N-acylamino acid and/or a salt thereof, in particular with a glutamic acid derivative and/or a salt thereof, or with a fatty ester, in particular with a jojoba ester.

According to a more particularly preferred embodiment, the pigments may be coated with an N-acylamino acid and/or a salt thereof, in particular with a glutamic acid derivative and/or a salt thereof, especially a stearoyl glutamate, for instance aluminum stearoyl glutamate.

Examples of coated pigments according to the invention that may be mentioned more particularly include titanium dioxides and iron oxide coated with aluminum stearoyl glutamate, sold, for example, under the reference NAI by Miyoshi Kasei.

Pigments not Coated with a Hydrophobic Compound

As stated previously, a composition may also contain pigments not coated with a lipophilic or hydrophobic compound.

These other pigments may be coated with a hydrophilic compound or uncoated.

These pigments may be mineral pigments especially as defined previously.

These pigments may also be organic pigments.

The term “organic pigment” refers to any pigment that satisfies the definition in Ullmann's Encyclopedia in the chapter on organic pigments. The organic pigment may in particular be chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane and quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indolic or phenolic derivatives as described in patent FR 2 679 771.

These pigments may also be in the form of composite pigments as described in patent EP 1 184 426. These composite pigments may especially be composed of particles comprising an inorganic core at least partially covered with an organic pigment and at least one binder for fixing the organic pigments to the core.

The pigment may also be a lake. The term “lake” is intended to mean insolubilized dyes adsorbed onto insoluble particles, the assembly thus obtained remaining insoluble during use.

The inorganic substrates onto which the dyes are adsorbed are, for example, alumina, silica, calcium sodium borosilicate, calcium aluminum borosilicate and aluminum.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (0119 140), D&C Yellow 6 (CI 15 985), D&C Green (CI 61 570), D&C Yellow 1 0 (CI 77 002), D&C Green 3 (CI 42 053), D&C Blue 1 (CI 42 090).

An example of a lake that may be mentioned is the product known under the name D&C Red 7 (0115 850:1).

Nature of the Hydrophilic Coating

As stated previously, these other pigments may be coated with a hydrophilic compound.

Said hydrophilic compound for surface-treating a pigment in order to optimize its dispersion in the gelled aqueous phase is more particularly chosen from biological polymers, carbohydrates, polysaccharides, polyacrylates and polyethylene glycol derivatives.

As examples of biological polymers, mention may be made of polymers based on monomers of carbohydrate type.

More particularly, mention may be made of biosaccharide gum; chitosans and derivatives thereof, such as butoxy chitosan, carboxymethyl chitosan, carboxybutyl chitosan, chitosan gluconate, chitosan adipate, chitosan glycolate, chitosan lactate, etc.; chitins and derivatives thereof, such as carboxymethyl chitin, chitin glycolate; cellulose and derivatives thereof such as cellulose acetate; microcrystalline cellulose; distarch phosphate; sodium hyaluronate; soluble proteoglycans; galacto-arabinans; glycosaminoglycans; glycogen; sclerotium gum; dextran; starch and derivatives thereof; and mixtures thereof.

Examples of carbohydrates that may especially be mentioned include polyhydroxyaldehydes and polyhydroxy ketones of general formula: Cₓ(H₂O)_{y} in which x and y may range from 1 to 1 000 000.

The carbohydrates may be monosaccharides, disaccharides or polysaccharides.

Examples of carbohydrates that may especially be mentioned include amylodextrins, beta-glucans, cyclodextrins, modified corn starch, glycogen, hyaluronic acid, hydroxypropylcyclodextrin, lactose, maltitol, guanosine, glyceryl starch, Triticum vulgare starch, trehalose, sucrose and derivatives thereof, raffinose and sodium chondroitin sulfate.

C₁-C₂₀ alkylene glycols or C₁-C₂₀ alkylene glycol ethers, alone or used in combination with tri-C₁-C₂₀-alkylsilanes, may also be used as surface treatment agents.

Examples that may be mentioned include pigments surface-treated with PEG alkyl ether alkoxysilane, for instance pigments treated with PEG-8-methyl ether triethoxysilane sold by the company Kobo under the name SW pigments.

Silicones such as dimethicones bearing hydrophilic groups, also known under the name dimethicone copolyols or alkyl dimethicone copolyols, may also be suitable for use in the invention as surface treatment agents. In particular, such dimethicones may comprise, as repeating units, C₁-C₂₀ alkylene oxides, such as ethylene or propylene oxides.

An example that may be mentioned is the pigment treated with PEG-12-dimethicone, sold by the company Sensient Corporation under the name LCW AQ Pigment.

The amount of pigments coated with at least one hydrophilic compound and/or of uncoated pigments is especially conditioned by the intended use of the cosmetic composition under consideration, and the adjustment of this amount obviously falls within the competence of the composition formulator.

According to one particularly preferred embodiment, the composition of the invention comprises at least one pigment coated with at least one lipophilic or hydrophobic compound, in particular iron oxide and/or titanium dioxide pigments coated with at least one lipophilic or hydrophobic compound.

According to a particularly preferred embodiment, the pigments may be coated with an N-acylamino acid and/or a salt thereof, in particular with a glutamic acid derivative and/or a salt thereof, or with a fatty ester, in particular with a jojoba ester.

According to a more particularly preferred embodiment, the pigments may be coated with an N-acylamino acid and/or a salt thereof, in particular with a glutamic acid derivative and/or a salt thereof, especially a stearoyl glutamate, for instance aluminum stearoyl glutamate.

Examples of coated pigments according to the invention that may be mentioned more particularly include titanium dioxides and/or iron oxides coated with aluminum stearoyl glutamate, sold, for example, under the reference NAI by Miyoshi Kasei.

According to one particularly preferred mode, the composition of the invention comprises

a) at least one silicone/urea copolymer of the copolymer of bis-aminopropyl dimethicone and of isophorone diisocyanate type with the INCI name: BIS-AMINOPROPYL DIMETHICONE/IPDI COPOLYMER; and

b) at least one C₈-C₁₆ branched volatile alkane such as isododecane, isodecane, isohexadecane and mixtures thereof and more particularly isododecane;

c) at least one MQ silicone resin, in particular of trimethylsiloxysilicate type; and

d) a monoalcohol chosen from ethanol, isopropanol, propanol, butanol and mixtures thereof, and more particularly isopropanol.

Even more particularly, said composition is anhydrous.

Cosmetic Compositions

The present invention also relates to a cosmetic composition comprising, in a physiologically acceptable medium, a composition as defined above.

The term “physiologically acceptable medium” is intended to denote a medium that is particularly suitable for the application of a composition of the invention to the skin.

The physiologically acceptable medium is generally adapted to the nature of the support onto which the composition has to be applied, and also to the appearance under which the composition has to be packaged.

According to one particular form of the invention, the composition will be anhydrous.

For the purposes of the present invention, the term “anhydrous” refers to a composition comprising a content of less than or equal to 2% by weight, preferably less than or equal to 1% and more preferentially less than 0.5% by weight of water relative to the total weight of said composition, or is even free of water. Where appropriate, such small amounts of water may especially be introduced by ingredients of the composition that may contain residual amounts thereof.

Additives

The compositions according to the invention may in addition comprise additives commonly used in care and/or makeup products, such as:
active agents such as vitamins, for example vitamins A, E, C, B₃;
moisturizing agents;
sunscreens;
fillers;
additional colorants;
fragrances;
preservatives;
and mixtures thereof.

It is a matter of routine practice for those skilled in the art to adjust the nature and the amount of the additives present in the compositions in accordance with the invention such that the desired cosmetic properties thereof are not thereby affected.

Fillers

The compositions in accordance with the invention may also comprise at least one filler, of organic or mineral nature, which can especially give them complementary properties of matteness, coverage, persistence and/or improved stability.

The term “filler” should be understood as meaning colorless or white solid particles of any form, which are in an insoluble form dispersed in the medium of the composition. These particles, of mineral or organic nature, give body or rigidity to the composition and/or softness and uniformity to the makeup.

The fillers used in the compositions according to the present invention may be in lamellar, globular or spherical form, in the form of fibers or in any other intermediate form between these defined forms.

The fillers according to the invention may or may not be surface-coated, and in particular they may be surface-treated with silicones, amino acids, fluorinated derivatives or any other substance which promotes the dispersion and the compatibility of the filler in the composition.

Examples of mineral fillers that may be mentioned include talc, mica, silica, hollow silica microspheres, kaolin, calcium carbonate, magnesium carbonate, hydroxyapatite, boron nitride, glass or ceramic microcapsules, or composites of silica and of titanium dioxide, for instance the TSG series sold by Nippon Sheet Glass.

Examples of organic fillers that may be mentioned include polyamide powders (Nylon® Orgasol from Atochem), polyethylene powders, polymethyl methacrylate powders, polytetrafluoroethylene (Teflon) powders, acrylic acid copolymer powders (Polytrap from the company Dow Corning), lauroyl lysine, hollow polymer microspheres such as those of polyvinylidene chloride/acrylonitrile, for instance Expancel (Nobel Industrie), hexamethylene diisocyanate/trimethylol hexyllactone copolymer powder (Plastic Powder from Toshiki), silicone resin microbeads (for example Tospearl from Toshiba), synthetic or natural micronized waxes, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, Polypore® L 200 (Chemdal Corporation), powders of crosslinked elastomeric organopolysiloxane coated with silicone resin, especially with silsesquioxane resin, as described, for example, in patent U.S. Pat. No. 5,538,793, polyurethane powders, in particular powders of crosslinked polyurethane comprising a copolymer, said copolymer comprising trimethylol hexyllactone. It may in particular be a hexamethylene diisocyanate/trimethylol hexyllactone polymer. Such particles are especially commercially available, for example, under the name Plastic Powder D-400® or Plastic Powder D-800® from the company Toshiki, and mixtures thereof. It may also be cellulose powder, such as the product sold by Daito in the Cellulobeads range.

Additional Colorants

A composition according to the invention may also comprise at least one additional colorant, preferably in a proportion of at least 0.01% by weight relative to the total weight of the composition.

For obvious reasons, this amount is liable to vary significantly with regard to the intensity of the desired color effect and of the color intensity afforded by the colorants under consideration, and its adjustment clearly falls within the competence of those skilled in the art.

The additional colorants that are suitable for the invention are liposoluble.

For the purposes of the invention, the term “liposoluble colorant” is intended to mean any natural or synthetic, generally organic compound, which is soluble in an oily phase or in solvents that are miscible with a fatty substance, and which is capable of imparting color.

As liposoluble dyes that are suitable for use in the invention, mention may be made especially of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

Moisturizing Agent

A “moisturizing agent” is intended to mean, according to the present invention, any compound capable of penetrating into the stratum corneum and of keeping the latter moisturized.

The moisturizing agents that are usable according to the invention are especially chosen from polyols, urea and its derivatives, such as notably hydroxyalkyl urea, in particular hydroxyethyl urea such as the product sold under the trade name Hydrovance® by the company Akzo Nobel, hyaluronic acid, glycine, β-alanine, taurine, trimethyl glycine, and mixtures thereof.

For the purposes of the present invention, a “polyol” should be understood to be any organic molecule comprising at least two free hydroxyl groups.

According to one particular form, the polyol may be chosen from sugars such as trehalose, mannitol, xylitol, sorbitol, and mixtures thereof.

Preferably, a polyol according to the present invention is present in liquid form at ambient temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those in particular containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols, such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols and mixtures thereof.

According to a particular mode, the composition of the invention may comprise at least propylene glycol and/or glycerol.

The moisturizing agent(s) are preferably present in the composition in a content ranging from 1% to 20%, better still from 3% to 15% by weight, preferably from 5% to 10% by weight, relative to the total weight of said composition.

Applications

According to one embodiment, a composition of the invention may advantageously be in the form of a composition for caring for the skin and/or keratin fibers, of the body or of the face, in particular of the face.

According to another embodiment, a composition of the invention may advantageously be in the form of a composition for making up keratin materials, in particular the skin of the body or of the face, in particular of the face.

Thus, according to a sub-mode of this embodiment, a composition of the invention may advantageously be in the form of a makeup base composition.

A composition of the invention may advantageously be in the form of a foundation.

According to another sub-mode of this embodiment, a composition of the invention may advantageously be in the form of a composition for making up the skin and especially the face. It may thus be an eyeshadow or a face powder.

Such compositions are especially prepared according to the general knowledge of those skilled in the art.

Throughout the description, including the claims, the expression “comprising a” should be understood as being synonymous with “comprising at least one”, unless otherwise specified.

The expressions “between . . . and . . . ” and “ranging from . . . to . . . ” should be understood as being inclusive of the limits, unless otherwise specified.

The invention is illustrated in greater detail by the examples presented below. Unless otherwise indicated, the amounts shown are expressed as weight percentages.

### EXAMPLES

| | INVENTION | COMPARATIVE | COMPARATIVE |
|---|---|---|---|
| INGREDIENTS | EXAMPLE 1 | EXAMPLE 2 | EXAMPLE 3 |
| BIS-AMINOPROPYL | 10% | — | 10% |
| DIMETHICONE (BELSIL UD 80 ® - | | | |
| WACKER) | | | |
| BIS-HYDROXYPROPYL | — | 10% A.M* | — |
| DIMETHICONE/SMDI | | | |
| COPOLYMER (SILMER UR-5050 ®- | | | |
| SILTECH CORPORATION) | | | |
| TRIMETHYLSILOXYSILICATE | 15% | 15% | 15% |
| (BELSIL TMS 803 ® - WACKER) | | | |
| ISOPROPYL ALCOHOL | 5% | — | — |
| PIGMENT | 20% | 20% | 20% |
| ISODODECANE | qs 100% | qs 100% | qs 100% |
| (PERMETHYL 99 ® A - | | | |
| PERMETHYL) | | | |

*A.M: Active Material

Procedure: the ingredients are mixed for 2 hours at 25° C. with a Rayneri mixer.

The compositions are applied to the skin. After drying (tests carried out after 1 hour of drying at ambient temperature), the following results are obtained:

The composition of exemplary embodiment 1 according to the invention forms a deposit after application and drying on the skin which has an excellent wear and transfer-free property on the skin and which is not tacky.

The composition of comparative example 2 forms a deposit after application and drying on the skin which has a prohibitively tacky nature.

The composition of comparative example 3 cannot be produced because the silicone/polyurea copolymer has not been dissolved.

1. A composition, especially for coating keratin materials, comprising:
a cosmetically acceptable medium:
a) at least one silicone/polyurea copolymer and/or a silicone/polyurethane copolymer and/or a silicone/polyurea/polyurethane copolymer of formula (I): 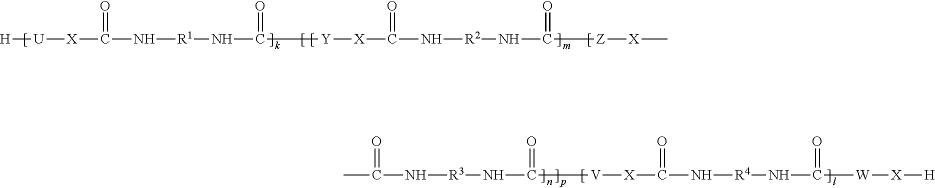
wherein:
the radicals R¹, R², R³, R⁴, which are identical or different, represent a linear or branched, acyclic or cyclic, aliphatic or aromatic C₆-C₂₀ divalent hydrocarbon-based group;
k and 1 are numbers ranging from 0 to 100,
m is a number ranging from 0 to 100,
n and p are numbers ranging from 1 to 100,
X denotes an oxygen (O) or nitrogen (N) atom,
when X is an oxygen atom (O), m ranges from 1 to 100,
when X is a nitrogen atom (N), m ranges from 0 to 100,
Y denotes the following formula (II): 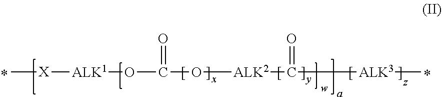
wherein
a is a number ranging from 1 to 100,
w is a number ranging from 0 to 100,
x, y and z, which are identical or different, are 0 or 1, and
when x=0, then w=1 and y=1 and z=1
when x=1, then y=0 and z=0
X represents an oxygen atom (O) or a nitrogen atom (N),
ALK¹, ALK², ALK³, which are identical or different, represent a linear or branched, acyclic or cyclic, aliphatic or aromatic C₁-C₂₀ divalent hydrocarbon-based group,
Z denotes the formula (III) 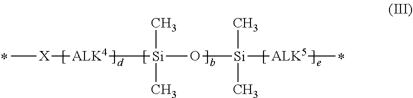
in which:
b is a number ranging from 1 to 1000,
d and e, which are identical or different, are 0 or 1, and
X represents an oxygen (O) or nitrogen (N) atom,
ALK⁴, ALK⁵, which are identical or different, represent a *—(CH₂)_{c}—* group as defined previously;
U, V, W represent the formula (II) or the formula (III) as defined previously; and
b) at least one volatile alkane; and
c) at least one monoalcohol comprising from 2 to 8 carbon atoms; and
d) at least one silicone resin.

2. The composition of in claim 1, wherein the silicone/polyurea copolymer is obtained by copolymerization of a diisocyanate with an α,ω-aminopolysiloxane.

3. The composition of claim 1, wherein the silicone/polyurethane copolymer is obtained by copolymerization of a diisocyanate with an α,ω-hydroxypolysiloxane and a diol selected from the group consisting of polyether diols, polyester diols and polycarbonate diols.

4. The composition of claim 1, wherein the copolymer is a silicone/polyurea/urethane copolymer obtained by copolymerization of a diisocyanate with an α,ω-aminopolysiloxane and an α,ω-hydroxypolysiloxane.

5. The composition of claim 1, wherein the silicone/polyurea copolymer is a copolymer of bis-aminopropyl dimethicone and isophorone diisocyanate with the INCI name BIS-AMINOPROPYL DIMETHICONE/IPDI COPOLYMER.

6. The composition of claim 1, wherein the copolymer(s) is (are) present in the composition in a content ranging from 1% to 20% by weight relative to the total weight of said composition.

7. The composition of claim 1, wherein the volatile alkane(s) is (are) a branched volatile alkane.

8. The composition of claim 1, wherein the volatile alkane(s) are present in the composition in concentrations ranging from 35% to 95% by weight relative to the total weight of the composition.

9. The composition of claim 1, wherein said monoalcohol is at least one selected from the group consisting of ethanol, isopropanol, propanol, butanol and mixtures thereof.

10. The composition of claim 1, wherein the amount of monoalcohol(s) ranges from 2% to 20% by weight relative to the total weight of said composition.

11. The composition of claim 1, wherein the resin is a resin of MQ type.

12. The composition of claim 1, wherein the silicone resin is present in the composition in a resin solids content ranging from 2% to 20% by weight relative to the total weight of the composition.

13. The composition of claim 1, further comprising at least one pigment selected from iron oxides and/or titanium dioxides optionally coated with at least one lipophilic or hydrophobic compound.

14. The composition of claim 1, further comprising at least 5% by weight of pigment(s) relative to the total weight of said composition.

15. The composition of claim 14, wherein the pigments are mineral pigments coated with at least one lipophilic or hydrophobic compound.

16. The composition of claim 1, in the form of an anhydrous composition.

17. A process for coating keratin materials, comprising the application to the keratin materials of a composition of claim 1.
